(19) United States
(12) Patent Application Publication
(10) Pub. No.: US 2019/0331690 A1
(43) Pub. Date: Oct. 31, 2019

(54) BIOTHIOL DETECTION COMPOSITION COMPRISING REDOX REGULATION PROTENIN

(71) Applicant: INDUSTRY-UNIVERSITY COOPERATION FOUNDATION HANYANG UNIVERSITY, Seoul (KR)

(72) Inventors: Young-Pil KIM, Seoul (KR); Jin Oh LEE, Seoul (KR); Jin-Won LEE, Seoul (KR); Yoon Mo YANG, Seoul (KR); Tae-Wuk KIM, Gyeonggi-do (KR)

(73) Assignee: INDUSTRY-UNIVERSITY COOPERATION FOUNDATION HANYANG UNIVERSITY, Seoul (KR)

(21) Appl. No.: 16/347,321

(22) PCT Filed: Nov. 2, 2017
(86) PCT No.: PCT/KR2017/012344

§ 371 Date: May 3, 2019

(30) Foreign Application Priority Data
Nov. 4, 2016 (KR) 10-2016-0146831
Nov. 1, 2017 (KR) 10-2017-0144833

(51) Int. Cl. G01N 33/68

(52) U.S. Cl. CPC G01N 33/68; G01N 2458/30; G01N 2800/042; G01N 2800/28; G01N 2800/32

(57) ABSTRACT

The present invention relates to a biothiol detecting composition comprising a redox regulation protein, a method for detecting biothiols by using the same, and a biosensor/kit for detecting biothiols. The present invention provides the effect of rapidly measuring free biothiols in body fluids. In addition, relative content ratios and changes of total to free biothiols in body fluids can be detected in real time, which allows biothiols to be available as main indices of diseases through which prediction and warning can be made against various diseases. Further, because various redox stress changes associated with main diseases can be accounted for by variations of biothiols, the present invention can provide important technical, economical, and social values for the investigation of pathogenesis mechanisms and the diagnosis of diseases in the future.

### TECHNICAL FIELD

The present invention relates to a biothiol detecting composition including a redox-regulating protein, a biothiol detecting method using the composition, and a biosensor/kit for detecting a biothiol.

### BACKGROUND ART

A biothiol is a low molecular weight (LMW) thiol, which is different from a thiol such as cysteine present in a protein, and plays an important role in the resistance to oxidative stress and the regulation of physiological activity in vivo in all living organisms from bacteria to humans. Such biothiols are very sensitive to a redox reaction, are modified by a functional group such as SOH, SO₂H, SNO, or S—S, and thus serve as a regulatory switch for biomolecular activity. A biothiol is a material which is not only abundant in cells, but also widely detected in major human body fluids such as blood, urine, sweat, and tears. Biothiols present in the body fluids include cysteine (Cys), homocysteine (Hcy), glutathione (GSH), N-acetylcysteine (NAC), cysteamine (CA), γ-glutamylcysteine (γ-GluCys), cysteinylglycine (CysGly), N-acetylcysteine (N-AC), coenzyme A (CoA), coenzyme B (CoB), coenzyme M (CoM), bacillithiol (BacT), mycothiol (MyT), ergothioneine (ErT), and trypanothione (TrT). It has been reported that an in vivo change in concentrations of, particularly, Cys, Hcy and GSH among these biothiols is deeply related to various types of diseases, and all of the concentration ranges are different from each other [Non-Patent Documents 1 and 2]. In real plasma, while total concentrations (total oxidized and reduced amounts) of Hcy, Cys and GSH are in ranges of 6 to 20 μM, 150 to 350 μM and 4 to 10 μM, respectively, due to a high oxidizing condition in the plasma, almost all of Hcy and Cys are present in an oxidized form, and thus a free form is present in a very small amount, for example, about 0.2 μM or less in the case of Hcy and about 10 μM or less in the case of Cys. Similarly, GSH is also present in an excessive amount (>5 mM) in cells, but is rapidly converted in the plasma by γ-glutamyltransferase or the like, such that a free form is known to be present only at a concentration of 2 μM or less. The total concentrations of such biothiols and a relative change in concentration of a free form are known to be largely relevant to major diseases including a cardiovascular disease, a neurodegenerative disease, cancer, kidney dysfunction, diabetes mellitus, and bacterial and viral infections [Non-Patent Documents 3 to 7]. However, although such major biothiols, serving as common biomarkers for a disease, can be used as indicators that can detect an indicator capable of detecting abnormal responses in living organisms early, there is no method of effectively detecting the concentration of LMW biothiols due to a rapid redox process. Thereof, to date, LMW biothiols cannot be widely used.

Most of conventional standard analysis methods for measuring LMW biothiol level depend on High Performance Liquid Chromatography (HPLC), Gas Chromatography-Mass Spectrometry (GC-MS), or Capillary Electrophoresis (CE). However, in such analysis methods, there is a technical challenge in that LMW biothiols have to be reduced to measure only total amounts because the pretreatment of a body fluid sample is required with excessive time consumption and a cost issue. While various documents had disclosed that free LMW biothiols were measured through the addition of an antioxidant and a metal ion chelating agent during the pretreatment of bloodsample [Non-Patent Documents 8 and 9], even in this environment, it was difficult to exactly measure an amount of free LMW biothiols because of very fast oxidation of the free LMW biothiols for long analysis time.

As another method of measuring LMW biothiols, a commercially available antibody-based immunoassay kit has been widely used, but all antibodies used herein do not directly recognize free LMW biothiols, but recognize a form in which an oxidized biothiol is bound with a protein (for example, in the case of blood, serum albumin). Therefore, there is a limit in that dynamic changes of free LMW biothiols and total LMW biothiols cannot be detected.

Among currently reported methods, diverse variants of fluorescent dyes, such as rhodamine, fluorescein, BODIPY, cyanine, flavone, or coumarin, have been used as chemical probes for the detection of free LMW biothiols based on fluorescence change [Non-Patent Document 8]. With these methods, it is difficult to measure oxidized LMW biothiols bound to proteins through a disulfide form because these chemicals rapidly react with free thiols and induce the above-mentioned fluorescent change. Furthermore, synthetic chemicals including a benzene ring variant have very low solubility, are vulnerable to a change in pH, and are capable of reacting with free Cys present in a protein. Thus, there is a major limitation in specifically measuring only free LMW biothiols. Most of all, when the chemical probes are directly applied to body fluid samples, interference caused by a wide range of autofluorescence significantly limits reproducibility and accuracy, and thus the detection of free LMW biothiols by this method is mostly limited in intracellular fluorescence imaging.

Therefore, it is urgent to develop a novel technology to solve the above-described problems and to quickly and accurately measure contents of free and total LMW biothiols as a disease indicator in a body fluid.

### Non-Patent Documents

(Non-Patent Document 1) Persichilli, S., Gervasoni, J., Castagnola, M., Zuppi, C. & Zappacosta, B. A Reversed-Phase HPLC Fluorimetric Method for Simultaneous Determination of Homocysteine-Related Thiols in Different Body Fluids. Labmedicine 42, 657-662 (2011)
(Non-Patent Document 2) Fiskerstrand, T., Refsum, H., Kvalheim, G. & Ueland, P. M. Homocysteine and Other Thiols in Plasma and Urine—Automated-Determination and Sample Stability. Clin Chem 39, 263-271 (1993))
(Non-Patent Document 3) Seshadri, S. et al. Plasma homocysteine as a risk factor for dementia and Alzheimer's disease. New Engl J Med 346, 476-483, (2002)
(Non-Patent Document 4) Refsum, H., Ueland, P. M., Nygard, O. & Vollset, S. E. Homocysteine and cardiovascular disease. Annu Rev Med 49, 31-62 (1998)
(Non-Patent Document 5) Herzenberg, L. A. et al. Glutathione deficiency is associated with impaired survival in HIV disease. P Natl Acad Sci USA 94, 1967-1972 (1997).
(Non-Patent Document 6) El-Khairy, L., Ueland, P. M., Refsum, H., Graham, I. M. & Vollset, S. E. in Circulation Vol. 103 2544-2549 (2001).
(Non-Patent Document 7) Andersson, A., Lindgren, A., Arnadottir, M., Prytz, H. & Hultberg, B. Thiols as a measure of plasma redox status in healthy subjects and in patients with renal or liver failure. Clin Chem 45, 1084-1086 (1999).
(Non-Patent Document 8) Jung, H. S., Chen, X. Q., Kim, J. S. & Yoon, J. Recent progress in luminescent and colorimetric chemosensors for detection of thiols. Chem Soc Rev 42, 6019-6031, doi:Doi 10.1039/C3cs60024f (2013).

### DISCLOSURE

### Technical Problem

Therefore, as a result of the inventors attempting to solve the problems of a conventional method of detecting a biothiol, they developed a biothiol-detecting composition usinga redox-regulating protein, which simultaneously measuresfree and total LMW biothiols, and performs detection with a very small amount, thereby improving sensitivity and exhibiting excellent immediacy and storage stability, and thus the present invention was completed.

Accordingly, the present invention is directed to providing a biothiol-detecting composition, which includes a redox-regulating protein.

The present invention is also directed to providing a method of detecting LMWbiothiols using the composition.

The present invention is also directed to providing a biosensor using the composition.

The present invention is also directed to providing a biochip using the composition.

### Technical Solution

In one aspect, the present invention provides LWM biothiols detecting composition which includes a redox-regulating protein.

In another aspect, the present invention provides a method of detecting a biothiol using the composition.

In still another aspect, the present invention provides a biosensor using the composition.

In yet another aspect, the present invention provides a biochip using the composition.

### Advantageous Effects

A biothiol detecting composition according to the present invention can quickly measure a free form of a biothiol in a body fluid. In addition, the relative content ratio between the total and free LMW biothiols in the body fluid and the content change thereof can be detected in real-time, and thus the biothiol can be used as a major indicator of a disease, allowing the prediction and warning of various diseases. In addition, as changes in redox stress associated with major diseases can be explained by variations in LMW biothiols, it can provide important technical, economic and social values for the identification of a pathological mechanism of a disease and the diagnosis of a disease in the future.

### DESCRIPTION OF DRAWINGS

FIG. 1 is a schematic diagram illustrating the principle that an organic hydroperoxide regulator (OhrR) is dissociated from operator dsDNA by a LMW biothiol.

FIG. 2 is the result obtained by measuring real-time binding of OhrR to cysteine (Cys), homocysteine (Hcy) and glutathione (GSH) using fluorescence anisotrophy (FA).

FIG. 3 is the experimental result obtained by confirming the binding of OhrR protein with operator dsDNA using electrophoresis.

FIG. 4 is the experimental result obtained by measuring a process of dissociating an OhrR protein from operator dsDNA by a LMW biothiol without a fluorescent tag on the surface of a photosensor using a photorefractive index.

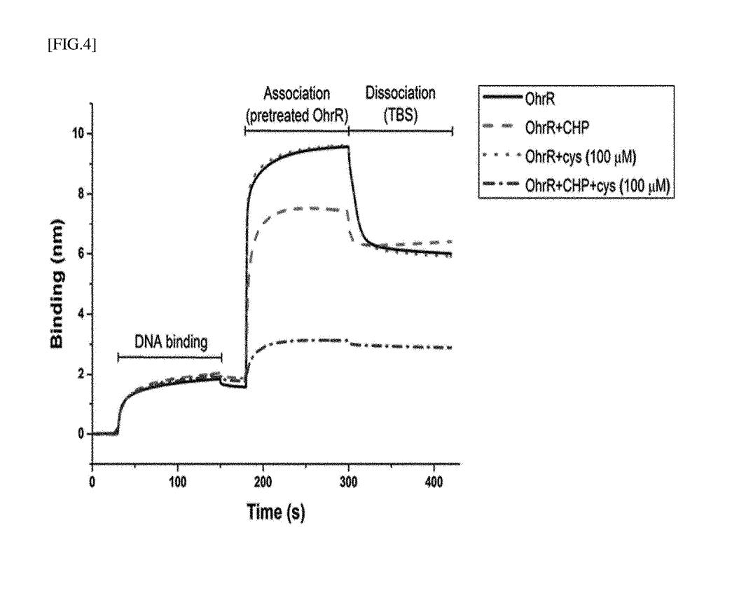

FIG. 5 is the result of confirming whether the association between OhrR and a LMW biothiol is quantitatively analyzed using MALDI-TOF MS.

FIG. 6 is the experimental result obtained by analyzing the relative amount of free LMW biothiols in a control and a comparative group in a mouse blood sample and a human blood sample using MALDI-TOF MS and OhrR.

FIG. 7 is the result obtained by investigating a real-time reaction of OhrR and Cys under a reducing condition using FA to confirm the possibility of measuring total LMW biothiols.

FIG. 8 is the result obtained by detecting free cysteine and total cysteine after cysteine at different concentrations is added to mouse blood using MALDI-MS and OhrR.

FIG. 9 is a schematic diagram illustrating a process of detecting LMW biothiols according to an exemplary embodiment of the present invention.

FIG. 10 is the result obtained by measuring fluorescence according to the method of detecting LMW biothiols, shown in FIG. 9.

FIG. 11 is the result obtained by measuring chemiluminescence according to the method of detecting LMW biothiols as shown in FIG. 9.

FIG. 12 is a schematic diagram illustrating signal amplification of DNA.

FIG. 13A is the result obtained by measuring chemiluminescence to confirm DNA signal amplification of FIG. 12.

FIG. 13B is the electrophoresis result to confirm DNA signal amplification of FIG. 12.

FIG. 14 is a schematic diagram of the configuration of a biochip for measuring the association between OhrR and dsDNA.

FIG. 15 is the result obtained by detecting LMW biothiols using the biochip of FIG. 14.

FIG. 16 is a schematic diagram of a strip-type biosensor of FIG. 9.

### MODES OF THE INVENTION

According to an exemplary embodiment of the present invention, a LMW biothiol detecting composition including a redox-regulating protein and a method of detecting a biothiol using the same are provided.

According to an exemplary embodiment of the present invention, a biothiol detecting composition which further includes DNA which binds to a redox-regulating protein, in addition to a redox-regulating protein, and a method of detecting a biothiol using the composition are provided.

The term “redox-regulating protein” used herein refers to all proteins whose activity is regulated by a redox reaction, and includes, representatively, an organic hydroperoxide regulator (OhrR) and a peroxide regulator (PerR), which are present in some species of bacteria such as B. subtilis, and an oxygen regulator (OxyR) present in some species of bacteria including E. coli. In addition, a redox-regulating protein more selectively reacting with a specific biothiol, by modifying an amino acid of a redox-regulating protein activation site by introducing protein engineering or screening for an orthologue protein present in a different type of an organism, may be included.

The redox-regulating protein may include a variant capable of regulating the binding affinity between a biothiol and DNA, or a protein tag-conjugated protein. Examples of the conjugate form may include fluorescent protein-OhrR, luminescent protein-OhrR, FLAG-OhrR, His6-OhrR, GSH-OhrR, and biotin-OhrR.

The term “biothiol” used herein refers to a LMW thiol having a molecular weight of 10 to 1,000 Da (preferably, 10 to 500 Da), and specifically, may be one or more selected from the group consisting of cysteine (Cys), homocysteine (Hcy), glutathione (GSH), N-acetylcysteine (NAC), cysteamine (CA), γ-glutamylcysteine (γ-GluCys), cysteinylglycine (CysGly), N-acetylcysteine (N-AC), coenzyme A (CoA), coenzyme B (CoB), coenzyme M (CoM), bacillithiol (BacT), mycothiol (MyT), ergothioneine (ErT) and trypanothione (TrT), but the present invention is not limited thereto.

In addition, a biothiol is a biomarker of various diseases associated with a cardiovascular disease, a neurodegenerative disease, cancer, kidney dysfunction, diabetes mellitus, and bacterial and viral infections, and an indicator capable of detecting abnormal responses in living organisms early.

The present invention includes a LMW biothiol detecting composition including a redox-regulating protein and DNA binding to the redox-regulating protein, and a method of detecting a biothiol using the composition.

According to an exemplary embodiment of the present invention, a biothiol may be detected by using the principle of binding/dissociation between a redox-regulating protein and its operator dsDNA.

In one example, the DNA may be represented by SEQ ID NO: 1 and/or SEQ ID NO: 2 used in the following examples, and any one that binds to a redox-regulating protein.

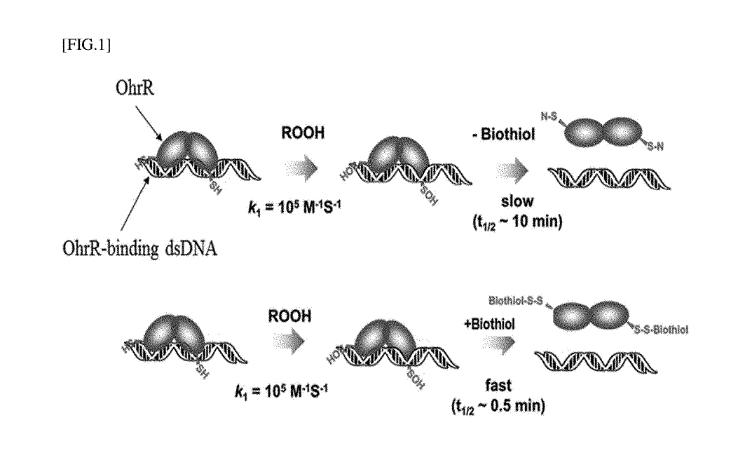

A principle of detecting the LMW biothiols is shown in FIG. 1 as an exemplary embodiment.

As a representative example of the redox-regulating protein, OhrR is a detection factor of an organic hydroperoxide (ROOH) present in bacteria, and OhrR is present as a homodimer and has one cysteine residue per monomer. While a cysteine residue is reduced (—SH), OhrR maintains a DNA-bound form (complex of OhrR and DNA), and OhrR is rapidly oxidized in the presence of an organic hydroperoxide (—SOH). The oxidized OhrR maintains its binding to dsDNA, and rapidly reacts with a biothiol in the presence of LMW biothiols, resulting in dissociation from dsDNA (generally, t_{1/2}˜0.5 min when the biothiol is contained at 10 μM or more; t_{1/2} means the time for a protein to be 50% dissociated from DNA). A dissociation rate varies according to the concentration and type of LMW biothiol. Meanwhile, OhrR forms a sulfenamide (—S—N—) at a relatively low rate by ROOH in the absence of a biothiol, and thus is slowly dissociated from dsDNA (t_{1/2}˜10 min).

The DNA binding to the redox-regulating protein may facilitate fluorescence, chemiluminescence and absorbance detections through conjugation of a fluorescent factor or a DNA-based enzyme (DNAzyme), or the DNA sequence may be amplified to improve reaction sensitivity by a signal amplification method.

A specific type of the fluorescent factor in the present invention is not particularly limited, and may be, for example, one or more selected from the group consisting of rhodamine and a derivative thereof, fluorescein and a derivative thereof, coumarin and a derivative thereof, acridine and a derivative thereof, pyrene and a derivative thereof, erythrosine and a derivative thereof, eosin and a derivative thereof, and 4-acetamido-4′-isothiocyanatostilbene-2,2′ disulfonic acid. More specific examples of the fluorescent materials that can be used in the present invention are as follows.

Rhodamine and derivatives thereof may include 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101, a sulfonylchloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, a terbium chelate derivative, an Alexa derivative, Alexa-350, Alexa-488, Alexa-547 and Alexa-647;

Fluorescein and derivatives thereof may include 5-carboxyfluorescein (FAM), 5-(4,6-dichlorotriazine-2-yl)aminofluorescein (DTAF), 2′7′-dimethoxy-4′5′-dichloro-6-carboxyfluorescein (6-FAM), fluorescein, fluorescein isothiocyanate, QFITC (XRITC), fluorescamine, IR144, IR1446, malachite green isothiocyanate, 4-methylumbelliferone, ortho cresol phthalein, nitrotyrosine, para-rosaniline, phenol red, B-phycoerythrin and o-phthalaldehyde;

Coumarin and derivatives thereof may include coumarin, 7-amino-4-methylcoumarin (AMC, coumarin 120), 7-amino-4-trifluoromethylcoumarin (coumarin 151), cyanosine, 4′-6-diaminidino-2-phenylindole (DAPI), 5′,5″-bromopyrogallol-sulfonphthalein (Bromopyrogallol Red), 7-diethylamino-3-(4′-isothiocyanatophenyl)-4-methylcoumarin diethylenetriamine pentaacetate, 4-(4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL) and 4-dimethylaminophenylazophenyl-4′-isothiocyanate (DAB ITC);

Acridine and derivatives thereof may include acridine, acridine isothiocyanate, 5-(2′-aminoethyl)aminonaphthalene-1-sulfonic acid (EDANS), 4-amino-N-[3-vinylsulfonyl)phenyl]naphthalimide-3,5disulfonate (LuciferYellow VS), N-(4-anilino-1-naphthyl)maleimide, anthranilamide and Brilliant Yellow;

Pyrene and derivatives thereof may include pyrene, pyrene butyrate, succinimidyl 1-pyrenebutyrate, and Reactive Red 4 (Cibacron® Brilliant Red 3B-A);

Erythrosine and derivatives thereof may include erythrosine B, erythrosine isothiocyanate and ethidium;

Eosin and derivatives thereof may include eosin and eosin isothiocyanate; and

4-acetamido-4′-isothiocyanatostilbene-2,2′ disulfonic acid is included.

The DNA-based enzyme may have one or more sequences selected from the group consisting of a peroxidase-mimicking DNAzyme having peroxidase characteristics [Wang Li et al. Insight into G-quadruplex-hemin DNAzyme/RNAzyme: adjacent adenine as the intramolecular species for remarkable enhancement of enzymatic activity. Nucleic Acids Research 44(15); 7373-7384 (2016)] and an RNA-cleaving DNAzyme [Meng Liu, Dingran Chang, and Yingfu Li. Discovery and Biosensing Applications of Diverse RNA-Cleaving DNAzymes, Accounts of Chemical Research, 50; 2273-2283 (2017)] among various DNA-based enzymes, but the present invention is not limited thereto.

According to the signal amplification method, a DNA sequence is amplified without PCR by binding a short DNA sequence to one end of a redox-regulating protein-binding DNA (combining single strand DNA templates capable of DNA amplification) and additionally reacting Hairpinl (HP1) and Hairpin 2 (HP2). The sequence and length of DNA, and a hairpin type are not limited as described in the following example (Table 1).

In addition, only DNA may be separated or bound to the surface of a bead, a nanoparticle or a chip by binding tags including a biotin group, an alkyne group, an azide group, a thiol group, and an amine group to the 5′ or 3′ end of the DNA sequence.

The term “biothiol detection” refers to measurement of a biothiol using redox regulation.

The biothiol measurement is performed by one or more selected from the group consisting of gel electrophoresis, fluorescence anisotropy, Matrix-Assisted Laser Desorption Ionization-Time-of-Flight Mass Spectrometry (MALDI-TOF MS), surface plasmon resonance (SPR), interferometry, and a bead measurement method.

Particularly, by using electrophoresis, the binding of a protein and DNA can be easily confirmed without specific equipment.

In addition, by using MALDI-TOF MS, the binding between a redox-regulating protein and a biothiol may be analyzed quantitatively according to the type of LMW biothiols, and free or total LMW biothiol level may be analyzed.

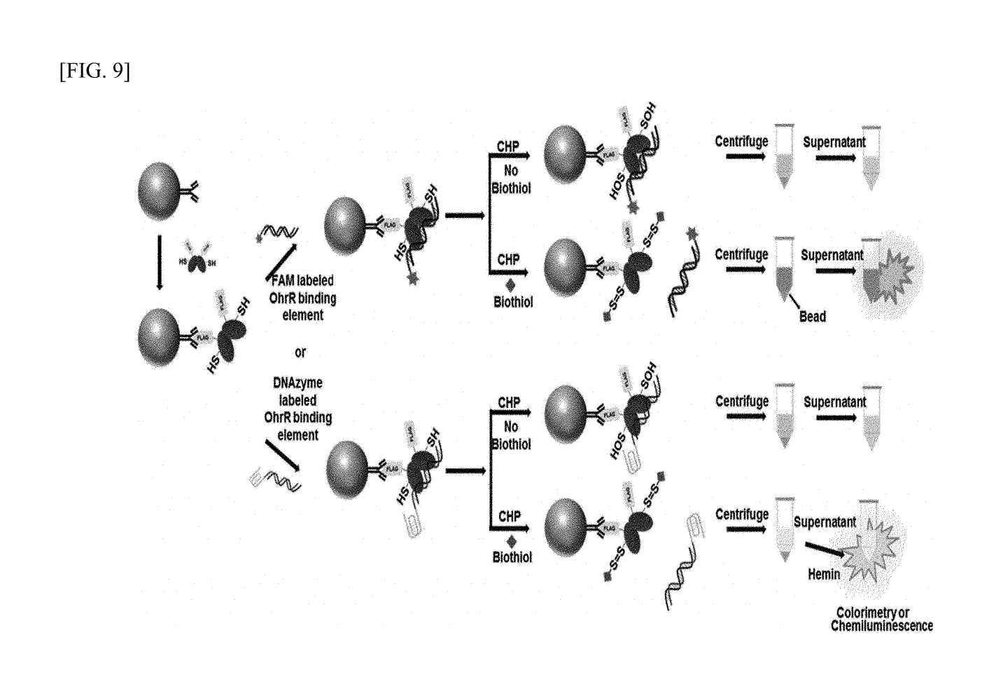

In addition, by using a bead measurement method, as shown in FIG. 9, a biothiol may be detected by fluorescence or chemiluminescence by linking a fluorescent tag or DNA-based enzyme with redox-regulating protein-binding DNA. Here, a FLAG tag, a His6 tag, a GSH tag, or a biotin tag may be bound to a redox-regulating protein, and the protein bound thereto detects a biothiol using the tag and affinity beads (FLAG affinity bead, NTA-bead, glutathione bead, or avidin-based bead).

The biothiol detecting composition according to the present invention may independently or simultaneously detect each of free and total LMW biothiol.

According to an exemplary embodiment of the present invention, when the total biothiol is detected, a reducing agent is further added to the composition. Since OhrR only binds to reduced free LMW biothiols, to detect total LMW biothiols in a sample, by rapidly reducing an oxidized biothiol using a reducing agent in the sample, it is possible to detect the total biothiol by OhrR. The reducing agent is specifically one or more selected from the group consisting of dithiothreitol (DTT), 2-mercaptoethanol and tris(2-carboxyethyl)phosphine (TCEP), but the present invention is not limited thereto.

The present invention also includes a biochip for detecting LMW biothiols, which includes the composition.

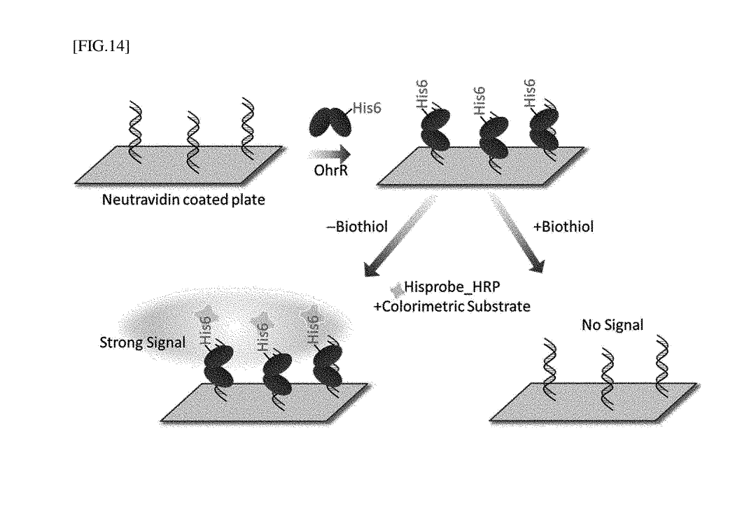

According to an exemplary embodiment of the present invention, as shown in FIG. 14, to form a complex with DNA capable of binding to a redox-regulating protein, the biochip is reacted with a redox-regulating protein on a DNA-immobilized plate. Here, since the redox-regulating protein is detached from DNA attached to the surface of the biochip using a redox-regulating protein bound with an affinity tag, rather than being labeled to DNA, in the presence of LMW biothiols, the biothiol is measured using a principle that a detection signal is reduced.

The present invention includes a biosensor for detecting LMW biothiols, which includes the composition.

According to an exemplary embodiment of the present invention, a strip-type biosensor for detecting LMW biothiols is included.

According to an exemplary embodiment of the present invention, a biosensor for detecting LMW biothiols includes three parts:

a sample introduction part which includes a fixing part capable of binding to a complex of a redox-regulating protein and DNA binding to the protein, and introduces a mixture of a sample and the complex;

a reaction part linearly spaced a predetermined distance apart from the sample introduction part, and in which DNA is dissociated after oxidation of the complex; and

a measurement part formed to measure LMW biothiols by transferring the dissociated DNA.

The fixing part may be combined with an antibody (e.g., FLAG tag-OhrR, Anti-FLAG antibody, or anti-His6 antibody) or a receptor (e.g., His6 tag-OhrR, NTA or Biotin-OhrR, or Strepavidin (including avidin and NeutrAvidin)).

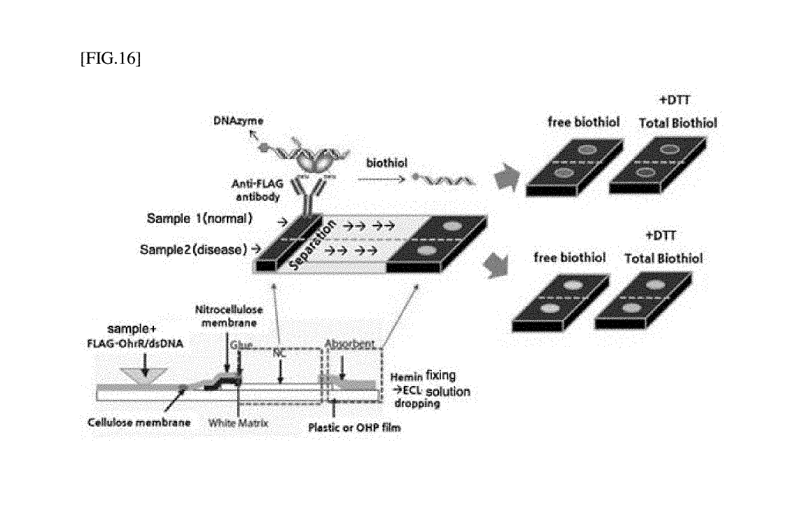

For example, such a strip-type biosensor for detecting a biothiol is shown in FIG. 16.

An oxidized/reduced amount of a LMW biothiol present in blood may be relatively measured. In the sample introduction part, when an antibody (e.g., an anti-FLAG antibody or an anti-His6 antibody) or an affinity receptor (e.g., an avidin series or NTA) is immobilized to a fixing part capable of binding with an OhrR-dsDNA complex, an OhrR-dsDNA mixture was dropped into a sample (e.g., plasma), the mixed solution flows to a right side due to the chromatographic principle and passes through the fixing part, and then the OhrR-dsDNA no longer moves and binds to the fixing part. Here, in the presence of a biothiol in a sample, dsDNA binding to OhrR is rapidly dissociated, such that only dsDNA moves to the right side, and reacts with the matrix by a DNAzyme present in the dsDNA in the measurement part. The biothiol may be detected by chemiluminescence.

When a biothiol is detected using a redox-regulating protein according to the present invention, compared with a conventional biothiol detection method, the method of the present invention has the distinctiveness and excellence as follows.

First, sensitivity can be significantly improved, and thus an amount of a sample for measurement may be reduced to a minimum. Since an OhrR monomer is bound with a biothiol at 1:1 (molar ratio), the macromolecule protein (e.g., OhrR) reacting with the biothiol may be measured, thereby improving reaction sensitivity (for example, the mass spectrometry of OhrR and OhrR+ LMW biothiol). In addition, when the method is designed to introduce a signal factor and an amplification factor to the DNA site binding to the protein, and regulate a DNA signal by binding or dissociation of the protein, higher sensitivity may be obtained, compared with the conventional measurement method (chromatography, immunoassay, or chemical sensor-based analysis). In addition, currently, when the OhrR protein is used, a free biothiol as well as a total biothiol can be measured with only about 1 to 2 μL of blood, thereby reducing an amount of a sample for analysis to a minimum.

Secondly, stability is excellent. The redox-regulating protein OhrR can be mass-expressed, and although it is a protein, due to a relatively small size (17 kD), it is not easily degraded at room temperature, and thus can be stored for a long time. Generally, unlike a peroxide sensor protein, since OhrR has a specific reaction with an organic hydroperoxide, it is difficult to be easily modified under an oxidizing condition (e.g., in the presence of oxygen and hydroperoxide) in a body fluid, thereby exhibiting excellent stability, and can be difficult to dissociate under a normal condition due to a much higher DNA binding strength (K_{d}=10⁻⁹ M or less) than a general transcription-promoting factor. For these reasons, when protein-DNA binding strength is utilized, a background signal may be maintained very low.

Thirdly, high reaction specificity is high. The redox-regulating protein OhrR only reacts with an LMW biothiol in the presence of an organic oxide and does not react with a thiol group present in a macromolecule protein, and therefore, only a LMW biothiol is able to be specifically detected.

Fourthly, free and total LMW biothiols can be simultaneously measured. Since the redox-regulating protein OhrR can stably form a mixed disulfide with a biothiol even in the presence of a high concentration of a reducing agent as well as having rapid reactivity with free LMW biothiols, when a sample is treated with a high concentration of a reducing agent, a total amount of a reduced biothiol may be quickly detected using the redox-regulating protein.

Fifthly, reaction time can be significantly reduced. There is one Cys residue in the redox-regulating protein OhrR, and a second order reaction rate between the Cys residue and a peroxide is about 10⁴ to 10⁵ M⁻¹s⁻¹. This rate is several ten thousand times or several hundred thousand times faster than a reaction rate (about 1 M⁻¹s⁻¹) between a Cys residue present in a general protein and a peroxide of free LMW biothiol, and a second order reaction rate between a biothiol and the redox-regulating protein is about 10³ M⁻¹s⁻¹, which is also several thousand times faster than that with a chemical probe (10⁻² to 10¹ M⁻¹s⁻¹). In addition, since the redox-regulating protein sensing a biothiol is immediately dissociated from DNA (50% dissociation time, t_{1/2}<0.5 min), it may react with a sample within several minutes, thereby rapidly inducing a resulting signal.

Lastly, as the redox-regulating protein can simply consist of a protein that can be easily mass-expressed in E. coli and a short oligo sequence, it is very effective for the constitution of a low-price chip or biosensor.

Hereinafter, the present invention will be described in detail with reference to examples. The following examples are merely provided to explain the present invention, and thus the scope of the present invention is not limited to the following examples. The examples are provided so that the disclosure of the present invention is complete and those of ordinary skill in the art fully understand the scope of the present invention, and the present invention is only defined by the appended claims.

### EXAMPLES

### Example 1: Confirmation of Reactivity Between OhrR Protein and Cys, Hyc or GSH

The real-time DNA binding activity of OhrR was measured using fluorescence anisotropy (FA).

<Experimental Conditions>

Buffer: 20 mM Tris (pH 8.0) 150 mM NaCl, 5% Glycerol (vol/vol)

DNA concentration: 50 nM

OhrR concentration: 300 nM

CHP concentration: 3 μM

Measurement time: measured every 10s

Measurement condition: ex 492 nm; slit width 15 nm, em 520 nm; slit width 20 nm, integration time 1 s

DNA Sequences:

Template DNA:
(SEQ ID NO: 1)
5′-TAC AAT TAA ATT GTA TAC AAT TAA ATT GTA-3′
Complementary DNA:
(SEQ ID NO: 2)
5′-TAC AAT TTA ATT GTA TAC AAT TTA ATT GTA-3′

OhrR sequence: directly cloned in B. subtilis strain.

(SEQ ID NO: 3)
MENKFDHMKLENQLCFLLYASSREMTKQYKPLLDKLNITYPQYLALLLLW
EHETLTVKKMGEQLYLDSGTLTPMLKRMEQQGLITRKRSEEDERSVLISL
TEDGALLKEKAVDIPGTILGLSKQSGEDLKQLKSALYTLL ETLHQKN

<Experimental Process>

The binding activity between OhrR and fluorescence (6FAM, 6-carboxyfluorescein)-labeled OhrR-binding DNA was measured every 10 seconds using an LS55 luminescence spectrometer (PerkinElmer) after dissolution in 3 mL of a buffer at a concentration under the experimental conditions. When OhrR binds to DNA, an anisotropy value (Anis) increases. However, when OhrR is dissociated from DNA, an anisotropy value decreases. In each experiment, three representative biothiols such as cysteine (Cys), homocysteine (Hcy) and glutathione (GSH) were treated at various concentrations (0, 1, 2, 4, 8, 16, 32, and 64 μM). While OhrR was bound to DNA in the presence of only a free biothiol, a real-time dissociation rate of OhrR from DNA was measured by treating a type of representative organic hydroperoxide, that is, cumene hydroperoxide (CHP) (treated at 300 sec).

<Experimental Result>

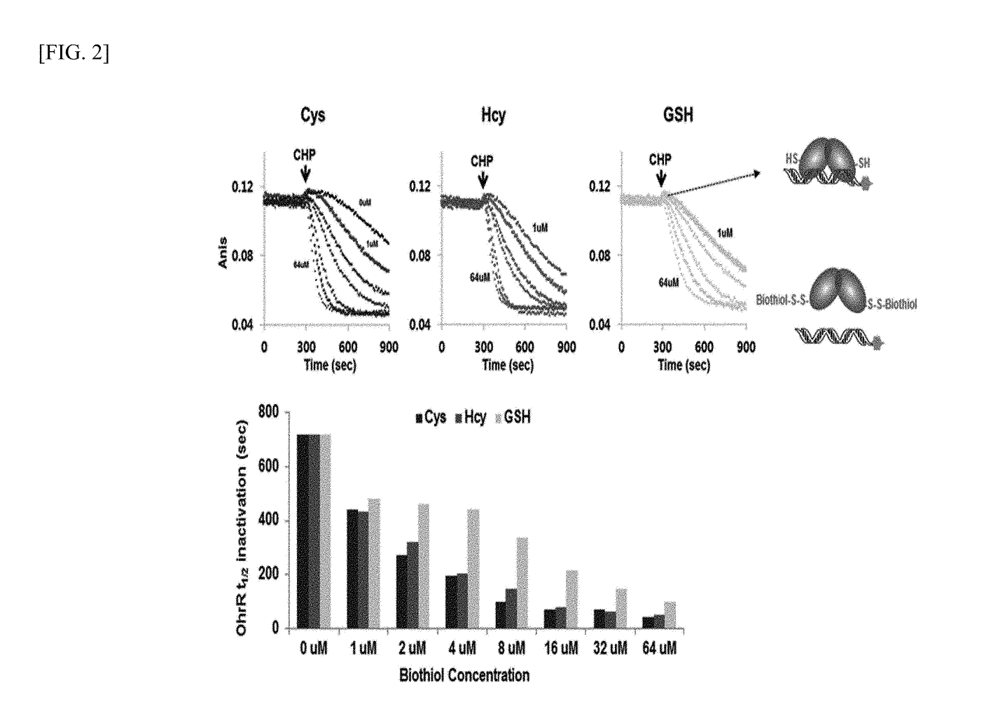

Referring to FIG. 2, it was confirmed the higher the concentration of the biothiol, the faster the dissociation rate of OhrR from DNA. The bar graph of FIG. 2 represents the time for an anisotropy value to be halved (time for half of OhrR to be dissociated from DNA), when OhrR was dissociated from DNA according to a concentration of a biothiol.

### Example 2: Confirmation of Binding Between OhrR Protein and Target DNA Using Electrophoresis

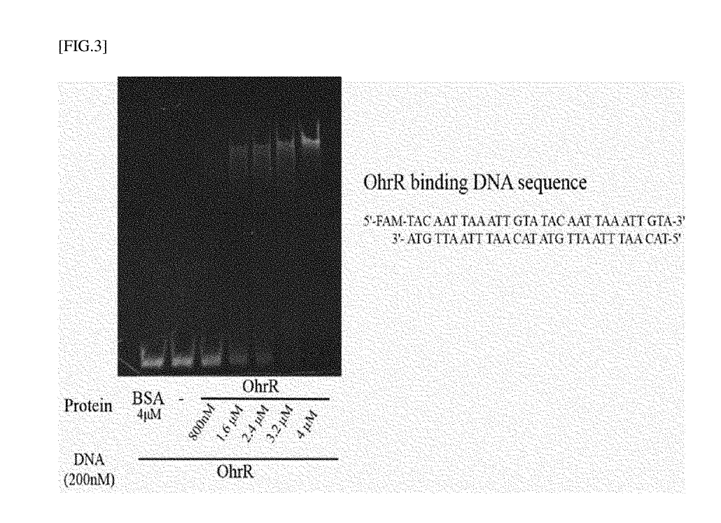

Fluorescent probe (FAM)-bound double strand (ds) DNA (200 nM) [SEQ ID NOs: 1 and 2] and OhrR at concentrations shown in FIG. 3 were mixed and reacted at room temperature for 30 minutes, and then a dsDNA band was detected using a fluorescence spectrometer (KIF-300, Korea Lab Tech, Korea) through electrophoresis (25 mA, 30 min) using a polyacrylamide gel (7%). It can be seen that the fluorescent band of dsDNA shifted upward from an OhrR concentration of about 1.6 μM (concentration about 8 fold higher than DNA concentration).

Unlike the FA measurement method described in Example 1, PAGE electrophoresis does not obtain a result of measuring protein-DNA binding in real time, and requires a large amount of proteins to bind OhrR and DNA (accordingly, an actual binding constant cannot be measured by this method), but this method can be used as a method for easily identifying binding without specific equipment.

### Example 3: Experiment of Measuring a Process of Dissociating Binding Between DNA and OhrR Protein without Fluorescent Tag Using Photorefractive Index

A binding degree between DNA and a protein on the surface of a photosensor (optical fiber) was measured using an instrument for biolayer interferometry (Blitz, Fortebio, USA).

<Experimental Conditions>

Binding buffer, washing buffer: TBS (Tris 20 mM, NaCl 150 mM)

DNA binding time, OhrR binding time: 2 min

Washing time: 30 sec

DNA concentration: 2.5 μM

OhrR concentration: 50 μM

CHP concentration: 100 μM

<Experimental Process>

First, biotin-binding dsDNA [SEQ ID NOs: 1 and 2] was dissolved in a TBS buffer at the above-mentioned concentration, bound to a streptavidin-coated photosensor (optical fiber) by flowing the resulting mixture for 120 seconds, and washed with a buffer, and then the OhrR protein was associated with dsDNA binding to the photosensor by additionally flowing the protein dissolved in a TBS buffer at the above-described concentration for 120 seconds (association). After the association was completed, by additionally flowing only a buffer for 120 seconds, the dissociation process of the DNA-OhrR complex was confirmed (dissociation).

<Experimental Result>

In biolayer interferometry, when a biomaterial (DNA or OhrR) is bound to the surface of a photosensor, a change in the surface refractive index of the photosensor is necessarily accompanied (the refractive index has a positive correlation with the concentration of a biomaterial), and the principle of converting the change in refractive index to a thickness is applied. OhrR was strongly bound with dsDNA and maintained a binding thickness (the value of the dissociation equilibrium of OhrR minus the dsDNA binding equilibrium) of about 4.3 nm. Similarly, even when any one of CHP and a biothiol is only mixed with the OhrR protein, and then the resulting mixture is flowed on DNA, it can be seen that OhrR effectively binds to DNA. On the contrary, in the presence of both CHP and a biothiol, an oxidation process of a DNA binding site of the OhrR protein by the biothiol accompanied, thereby confirming that the binding strength with DNA was rapidly reduced (thickness of about 1 nm). In other words, it can be confirmed that a concentration of the biothiol can be easily predicted in the presence of CHP by using the association of COhrR and DNA.

This method can be used as a method of effectively comparing reactivities to various types of biothiols and their concentrations without specific labeling of dsDNA or OhrR with a fluorophore on the surface of a photosensor.

### Example 4: Confirmation of OhrR-Biothiol Binding Using MALDI-TOF MS

It was confirmed whether it is possible to quantitatively analyze the binding of OhrR and a biothiol using a Matrix-Assisted Laser Desorption Ionization-Time-of-Flight Mass Spectrometry (MALDI-TOF mass spectrometer; MALDI-TOF MS).

1 mL of a 300 nM OhrR-containing buffer (20 mM Tris (pH 8.0), 100 mM NaCl, 5% Glycerol) was mixed with a biothiol (Cys, Hcy or GSH) at each concentration (0, 1, 2, 4, 8, 16, 32, or 64 μM). After 3 μM CHP reacted with each resulting mixture for 2 minutes, 110 μL of 100% TCA was treated to stop the oxidation reaction, and precipitate OhrR. To alkylate the precipitated OhrR, 50 mM iodoacetamide was treated, thereby blocking a cysteine residue of the reduced OhrR. The protein was isolated by sodium dodecyl sulphate-polyacrylamide gel electrophoresis (SDS-PAGE), and then a band corresponding to OhrR was cut out and treated with trypsin (37° C., 12 hours or more), followed by extraction from the gel. 0.5 μL of the extracted peptides were treated with 0.5 μL of a-cyano-4-hydroxycinnamic acid (CHCA) (in 50% acetonitrile, 1% TFA) and dehydrated, and then subjected to MALDI-TOF analysis. The MADI-TOF analysis was performed using a 4700 Proteomics Analyzer instrument (Applied Biosystems).

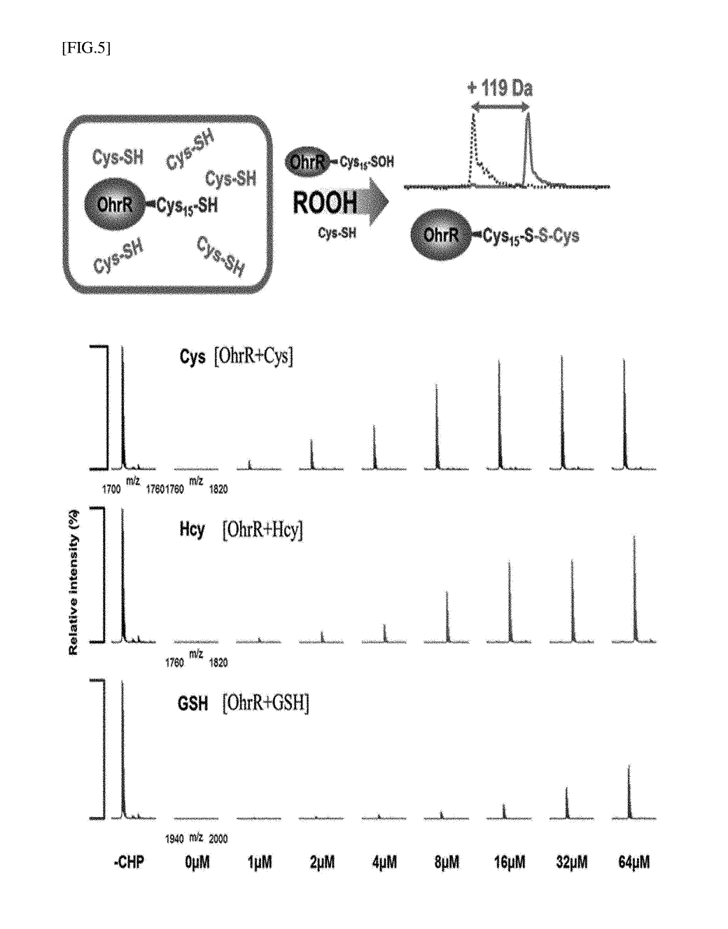

As shown in FIG. 5, as Cys, Hcy and GSH concentrations increased, an increase in m/z value corresponding to [OhrR+LMW biothiol] was detected, and although there was a slight difference in mass detection ability according to a LMW biothiol, it was confirmed that detection by MALDI-TOF MS is possible when the Cys, Hcy and GSH concentrations are approximately 1 μM or more, and Cys showed the highest detection signal level among the measured LMW biothiols.

### Example 5: Experiment for Free LMW Biothiol Analysis in Mouse/Human Blood Samples Using MALDI-TOF MS and OhrR

It was confirmed, by MALDI-TOF MS analysis, that the difference in free LMW biothiol level between mouse models (normal mouse and arteriosclerotic mouse model (ALDLR mouse) and human blood samples (blood of 25-year-old non-smoking male (human 1) and blood of 45-year-old smoking male (human 2)) can be analyzed using OhrR.

25 μM OhrR and 50 μM CHP were reacted with 2 μL of the plasma obtained from a mouse or human for 10 minutes (reaction in a total of 2.5 μL). After the reaction, 1 mL of 10% TCA was treated to precipitate proteins containing OhrR. To prevent further oxidation of the reduced cysteine of the precipitated OhrR, an alkylating agent, i.e., iodoacetamide, was treated at 50 mM and reacted with the reduced cysteine. After the isolation of the proteins by SDS-PAGE, the protein band corresponding to OhrR was cut out, and then treated with 0.2 μg of trypsin at 37° C. for over 12 hours. Peptides cleaved by the trysin were extracted from the gel through centrifugation. MALDI-TOF analysis was performed after 0.5 μL of the extracted peptides were treated with 0.5 μL of CHCA (in 50% Acetonitrile, 1% TFA) and dehydrated. The MADI-TOF analysis was performed using a 4700 Proteomics Analyzer instrument (Applied Biosystems).

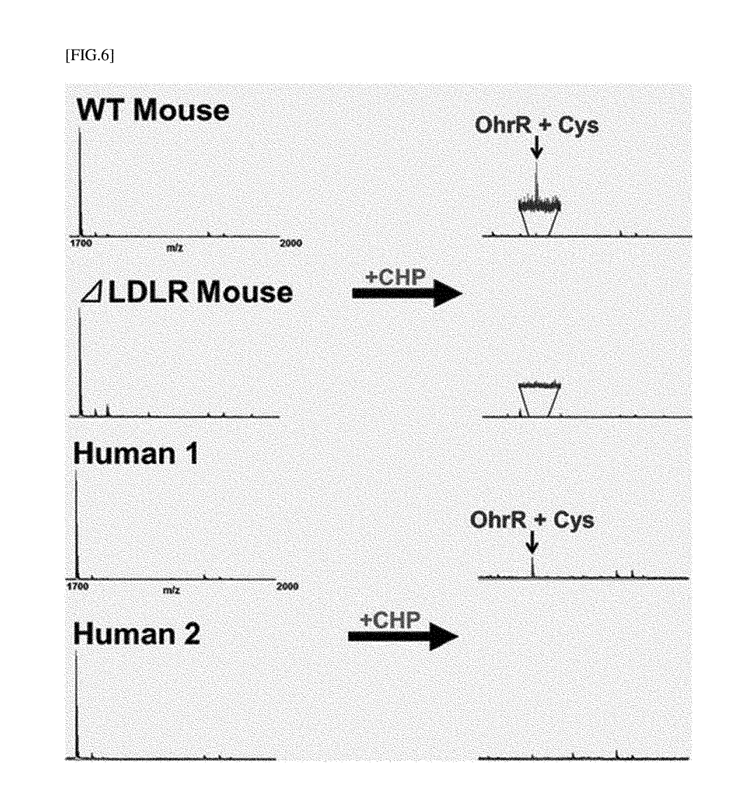

Referring to FIG. 6, it can be shown that, in the blood of the arteriosclerotic model mouse, the mass signal value of OhrR-Cys was reduced, compared with the blood of the normal mouse, and in the blood of the 45-year-old male smoker, the mass signal value of OhrR-Cys was reduced compared with the blood of the 25-year-old non-smoker. In other words, considering that when OhrR rapidly reacted with free LMW biothiols, the concentration of free LMW biothiol (particularly, free cysteine) present in the blood was relatively decreased in the mouse arteriosclerotic model and the smoker model. Thus, it was confirmed that the amount of free biothiols can be effectively compared and analyzed by mass spectrometry of OhrR with only a small amount of blood of patient groups with various diseases as described above.

### Example 6: Confirmation of Measurement of Total LMW Biothiols

### 1) Confirmation of FA Measurement

To confirm the possibility of the measurement of total LMW biothiols, the reactivity between OhrR and cysteine was investigated under the treatment of a reducing agent (dithiothreitol, DTT). FA measurement was performed in the same manner as described in the experimental process of Example 1, except that a Cys concentration of 0 or 1 mM and a CHP concentration of 30 μM were used.

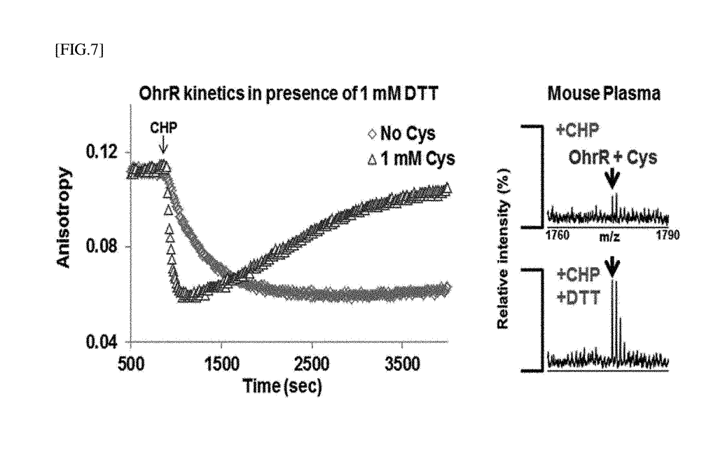

When DTT is treated, disulfides bonds of all types of oxidized LMW biothiols are reduced, thereby forming free forms. Afterward, when an oxide CHP is treated, oxidized OhrR rapidly reacted with reduced LMW biothiol again, thereby accelerating the dissociation rate of OhrR from DNA, and allowing an amount of total LMW biothiol to be measured (due to subsequent cyclization caused by internal binding between thiol molecules of DTT, DTT does not participate in additional reduction of LMW biothiols and not react with OhrR, either) [see the left drawing of FIG. 7].

### 2) Confirmation by MALDI-TOF Analysis

<Experiment Conditions (Mouse Plasma LMW Biothiol Detection Assay)>

OhrR concentration: 25 μM

CHP concentration: 50 μM

Plasma amount: 2 μL (purchased from Norma Mouse serum, Jackson ImmunoResearch)

DTT concentration: −/+1 mM (experiment progressed after 1-hour pretreatment)

<Experimental Process (Mouse Plasma LMW Biothiol Detection Assay)>

The plasma obtained from a mouse was reacted with −/+1 mM DTT at room temperature (RT; 23-25° C.) for 1 hour. Afterward, 2 μL of the plasma was reacted with 25 μM OhrR and 50 μM CHP for 10 minutes (reaction at a total of 2.5 μL). After the reaction, 1 mL of 10% TCA was treated so as to precipitate proteins containing OhrR. To prevent further oxidation of reduced cysteine of the precipitated OhrR, 50 mM of the alkylating agent, i.e., iodoacetamide, was reacted with the reduced cysteine. The proteins were isolated by SDS-PAGE, and the protein band corresponding to OhrR was cut out and treated with 0.2 μg of trypsin at 37° C. for 12 hours. The peptides cleaved by the trypsin were extracted from the gel by centrifugation. 0.5 μL of the extracted peptides were treated with 0.5 μL of CHCA (in 50% Acetonitrile, 1% TFA) and dehydrated, followed by performing MALDI-TOF analysis.

<Experimental Result>

It can be seen from the above-described experiment that LMW biothiols reduced in the presence of about 1 mM DTT rapidly reacted with OhrR in the presence of CHP (within about 5 minutes), and thereafter, the LMW biothiol can be dissociated from OhrR by DTT over time. For this reason, to detect the amount of a total LMW biothiol after the DTT treatment, the treatment may be performed within about 5 minutes after treatment of OhrR and CHP. As a result of analyzing the above-described conditions through MALDI-TOF MS, it was confirmed that, when mouse plasma was not treated with DTT, the amount of free cysteine may be measured, and when DTT was treated, the amount of total cysteine may be measured (see the right drawing of FIG. 7).

### Example 7: Confirmation of Simultaneous Detection of Free and Total LMW Biothiols

Free cysteine and total cysteine were detected using MALDI-TOF MS and OhrR, after various concentrations of cysteine were added to mouse blood. In other words, when the mouse serum was treated with DTT to reduce oxidized LMW biothiols, it was confirmed through MALDI-TOF MS analysis, in whicht the amount of LMW biothiols were analyzed as the OhrR increased.

<Experimental Conditions (Mouse Plasma Biothiol Detection Assay)>

OhrR concentration: 25 μM

CHP concentration: 50 μM

Cys concentration: 0/10/100 μM

Plasma amount: 2 μL (purchased from Norma Mouse serum, Jackson ImmunoResearch)

DTT concentration: −/+1 mM (experiment was performed after 1-hour pretreatment)

<Experimental Process (Mouse Plasma Biothiol Detection Assay)>

The experiment was performed in the same manner as the mouse plasma biothiol detection assay of Example 6, except that 0/10/100 μM free cysteine was used.

<Experimental Result>

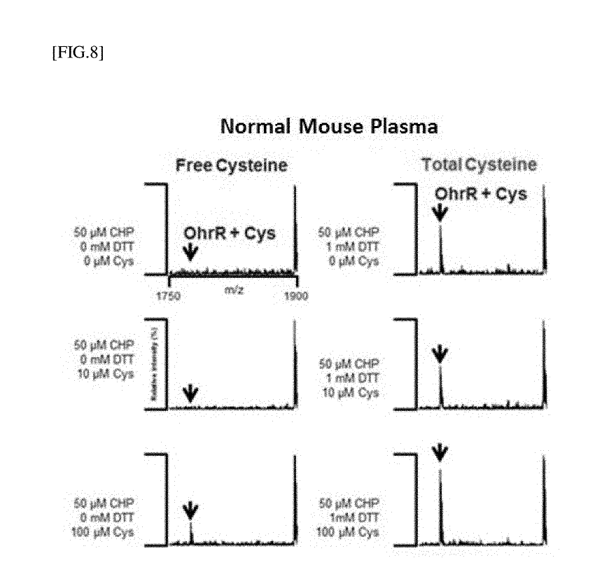

The result of free cysteine analysis for a normal mouse plasma without using DTT is shown in the left drawing of FIG. 8, and the result of total cysteine analysis after DTT was used is shown in the right drawing of FIG. 8. FIG. 8 shows the comparison of OhrR+cys (T3-Cys) mass analysis for the free cysteine analysis. The total cysteine was increased in the DTT-treated experimental group compared with the DTT-untreated experimental group, and the OhrR-Cys signal was relatively high. In addition, it can be seen that when cysteine was added randomly, the mass signal value of OhrR+Cys was found to be proportional to the concentration of added cysteine.

### Example 8: Confirmation of LMW Biothiol Detection Using Bead Measurement Method

This experiment is an example of measuring LMW biothiols using OhrR and DNA to realize a rapid and simple measurement method for LMW biothiols at the laboratory test level and the following Example 9 (labeling dsDNA with fluorophore tag) and Example 10 (labeling dsDNA with ssDNAzyme) are examples to which the present experimental process is commonly applied and only a different measurement method is applied.

First, 30 μL of M2 FLAG affinity beads (Sigma, A2220) were added to respective tubes corresponding to the number of samples, and FLAG-tagged OhrR (2 μM) was bound thereto for 1 hour to reach the total volume of 200 μL (when OhrR is bound to the His6 tag, NTA-Beads can also be used). To remove unbound OhrR, after spin-down, a supernatant was removed, fluorophore or DNAzyme-bound OhrR-binding dsDNA (100 nM, SEQ ID NOs: 1 and 2) was bound to the OhrR-bound beads for 30 minutes. After unbound dsDNA was removed by spin-down, 160 μL of a buffer (Tris Buffered Saline, TBS)->20 μL of LMW biothiols (by concentration)->20 μL of CHP (500 μM) were sequentially added to each tube, followed by shaking for 5 minutes. Afterward, 200 μL of a supernatant obtained by spin-down was transferred to a 96-well plate, and fluorescence or chemiluminescence was measured. The reducing agent (DTT)-untreated sample may be used for measuring free LMW biothiols, and the reducing agent-treated sample may be used for measuring total LMW biothiols [FIG. 9].

### Example 9: Fluorescence Measurement Using Fluorescence-Labeled dsDNA

OhrR-binding dsDNA was bound with a fluorescent factor FAM, and 200 μL of the final supernatant was transferred to a 96 well plate, followed by fluorescence measurement using a multi plate reader (Variokan, Thermo Scientific). In the fluorescence measurement, a fluorescent signal value at a 525 nm emission wavelength obtained at an excitation wavelength of 480 nm was obtained, and a fluorescence value of each well was corrected using a well only containing a buffer without a fluorescent factor as a reference value, followed by comparing these values.

FAM-labeled dsDNA [SEQ ID NOs: 1 and 2] was bound with OhrR, and when CHP and various concentrations of LMW biothiol were treated, a signal of the fluorescence-labeled dsDNA isolated from OhrR was measured. Compared with the treatment of CHP alone (control group) for the same period of time, it was confirmed that the fluorescence signal was quantitatively increased according to the concentration of CHP/LMW biothiol.

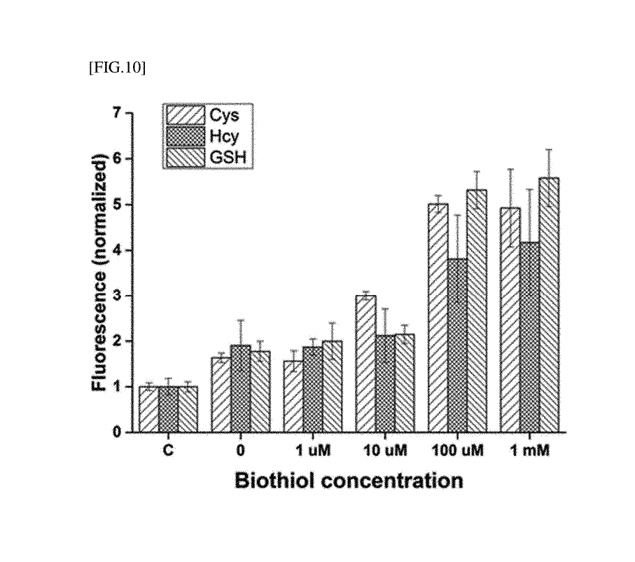

As the detection result obtained by adding a concentration of an arbitrary LMW biothiol shown in FIG. 10, although a detection amount according to the type of biothiol cannot be expected, through the present example, it can be shown that a total amount of LMW biothiol (amount of free LMW biothiol when DTT was not treated, and amount of oxidized LMW biothiol when DTT was treated) can be effectively detected.

### Example 10: Measurement of Chemiluminescence Using DNAzyme-Conjugated dsDNA

<Experimental Condition>

Buffer: TBS (Tris 20 mM, NaCl 150 mM)

OhrR concentration: 5 μM

DNA concentration: 100 nM

CHP, thiol reaction time: 5 min

dsDNA sequences (synthesized by IDT, USA)

Template DNA sequence:
[SEQ ID NO: 4]
5′- [symbol] [symbol] [symbol] TTT TTT
TTT TAC AAT TAA ATT GTA TAC AAT TAA ATT GTA-3'
(Italic/bold part represents a DNAzyme, and Italic
part represents T9-linker)
Complementary DNA sequence:
[SEQ ID NO: 5]
5′-biotin-TAC AAT TTA ATT GTA TAC AAT TTA ATT
GTA-3′

<Experimental Process>

200 μL of the DNAzyme (5′-GG GTT GGG CGG GAT GGG-3′ [SEQ ID NO: 6], synthesized by IDT)-bound OhrR-binding dsDNA was recovered from a supernatant, and 20 μL of a suspension of NeutrAvidin®-coated beads (Thermo Scientific, USA) was added thereto, followed by shaking at room temperature for 30 minutes. Afterward, washing was performed with a TBS (20 mM Tris, 150 mM NaCl, pH 7.4) buffer four times. To induce DNAzyme activity, 180 μL of a TBS buffer was mixed with 20 μL of 1 μM Hemin (Calbiochem, USA) (in TBS) solution to reach the final volume of 200 μL, the beads were precipitated, and then the resulting solution was left at room temperature under a dark condition for 15 minutes or more. Afterward, 200 μL of the bead-containing mixed solution was put into a tube and mounted in a luminometer (Trade name: Glo-Max 20/20, Promega, USA), and 100 μL of a mixture of ECL reaction solutions (G Healthcare, USA) A (Luminol+H₂O₂ solution, 50 μL) and B (enhancer, 50 μL) was added thereto, followed by immediately measuring a chemiluminescence intensity.

A ssDNAzyme (DNAzyme having properties of horseradish peroxidase) sequence was linked to the end of one strand of the OhrR-binding dsDNA sequence by a T9 linker [SEQ ID NO: 4], and biotin was introduced to the end of the other strand, thereby constructing a DNA probe [SEQ ID NO: 5]. The OhrR protein was used in the experiment after binding of a Flag peptide (DYKDDDDK, recombinant DNA was manufactured and directly expressed in a strain) [SEQ ID NO: 7] and expression/purification in E. coli. As described in Example 8, the OhrR protein was captured using anti-Flag antibody-bound agarose beads and interacted with the DNA probe, and then when CHP and cysteine were added into the sample, the DNA probe part is quickly dissociated. The dissociated DNA probe was present in a supernatant obtained by centrifugation, and the supernatant was reacted again with avidin-bound beads, washed and reacted with dsDNA and hemin, present in a bead-containing solution, followed by inducing strong chemiluminescence by a luminol reaction.

<Experimental Result>

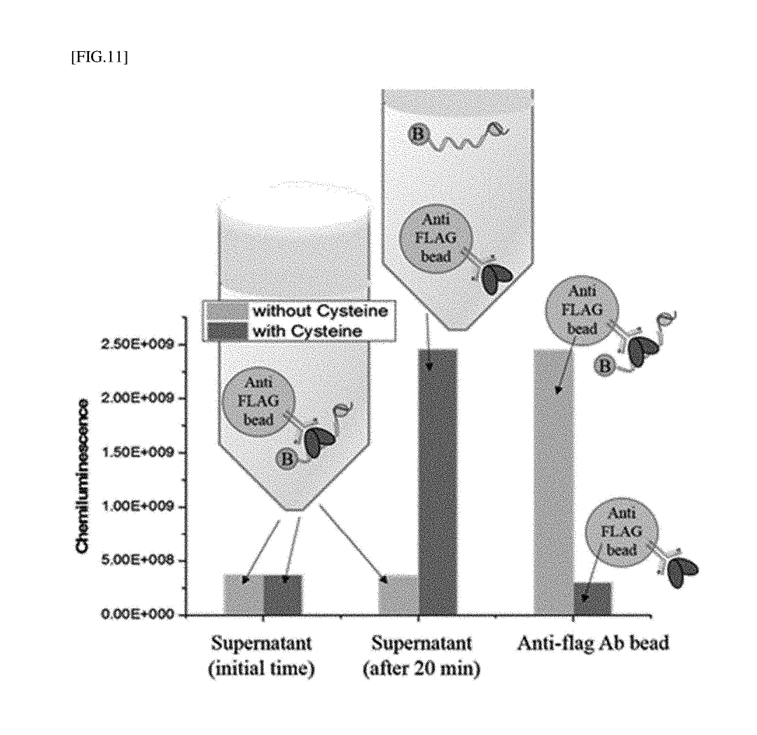

As a result, it was confirmed that, 20 minutes after a cysteine reaction, a chemiluminescence signal was effectively detected in a supernatant in which anti-Flag Ab beads are present (FIG. 11). As for the anti-Flag Ab beads, contrarily, under a cysteine-untreated condition, an excessive chemical signal was detected, confirming that the Flag-OhrR protein and a DNA probe are effectively dissociated in the presence of cysteine, and detected with a higher signal than fluorescence.

In other words, it was confirmed that DNA to which a DNAzyme specifically bound only in the presence of cysteine in a solution is dissociated from the OhrR protein, thereby exhibiting a DNAzyme signal (final concentrations of DNA probe and OhrR and cysteine are 1 riM, 1 riM, and 20 nM, respectively, chemiluminescence is measured 20 minutes after cysteine was added).

### Example 11: Method and Confirmation of DNA Signal Amplification

1) Signal Amplification

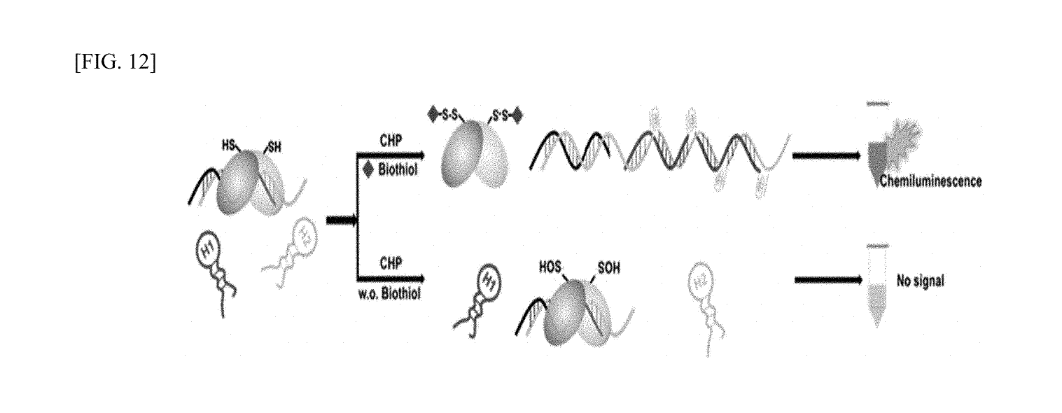

OhrR+F (template forward DNA) and OhrR+R (complementary reverse DNA) are dsDNAs constituting OhrR-binding DNA, and modified OhrR-F is dsDNA to which a short DNA sequence is additionally introduced for amplification (Table 1). When Hairpin 1 and Hairpin 2 are added to the final reaction product, the DNA sequence is amplified and detected by various methods (electrophoresis, fluorescence measurement, and luminescence measurement) [FIG. 12].

TABLE 1

| | | Length | | GC |
|---|---|---|---|---|
| Name | Sequence | (bp) | Tm | (%) |
| OhrR_F | 5′-TAC AAT TAA ATT GTA TAC AAT | 30 | 48 | 13 |
| | TAA ATT GTA-3′ | | | |
| | (SEQ ID NO: 8) | | | |
| OhrR_R | 5′-TAC AAT TTA ATT GTA TAC AAT | 30 | 48 | 13 |
| | TTA ATT GTA-3′ | | | |
| | (SEQ ID NO: 9) | | | |
| Modified | 5′-TAC AAT TAA ATT GTA TAC AAT | 42 | 60 | 31 |
| OhrR_F | TAA ATT GTA ACC ACC CAC CGG-3′ | | | |
| | (SEQ ID NO: 10) | | | |
| Hairpin | 5′-ACC ACC CAC CGG GGG TGG GTC | 45 | 76 | 64 |
| 1 | GTC TGC CGG TGG GTG GTT ACA | | | |
| | ATT-3′ (SEQ ID NO: 11) | | | |
| Hairpin | 5′-CAG ACG ACC CAC CCC CGG TGG | 61 | 77 | 67 |
| 2 | GTG GTA ATT GTA ACC ACC CAC | | | |
| | CGG GGG TGG GTG GGT GGGT-3′ | | | |
| | (SEQ ID NO: 12) | | | |

2 μL of each of the 2.5 μM template and 40 μM DNA of Hairpin 1 or 2, which were diluted in TBS, were added, and the final volume was adjusted to 20 μL with a TBS buffer. Subsequently, each sample was reacted at room temperature for 30 minutes or longer, 10 μL of the sample was transferred to a new tube, and then 10 μL of 1 μM Hemin and 80 μL of TBS were added thereto in order to adjust the final volume to 100 μL. Afterward, under a dark condition, the resulting product was reacted at room temperature for 30 minutes. In addition, to compare relative signal sizes, samples made with only hemin and TBS were additionally prepared. And then, by the same method as described in Example 10, DNAzyme activity was measured using a Glo-Max 20/20 single tube luminometer (Promega). The measurement result was expressed as a relative value based on the value obtained by reacting only hemin with an ECL solution.

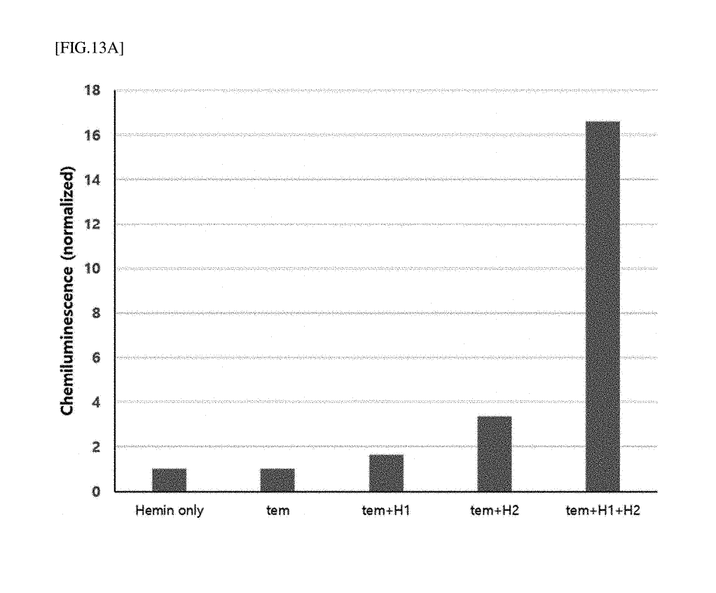

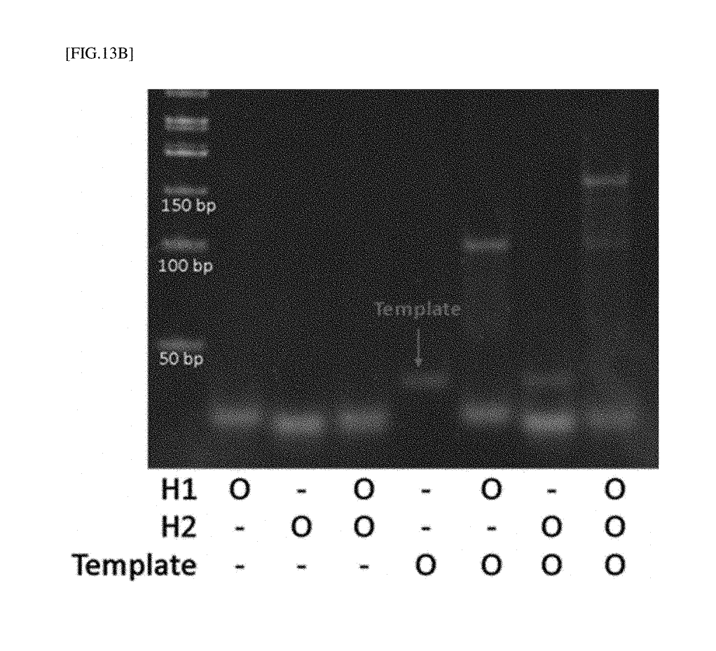

Compared with the case of using only one DNAzyme sequence at one end of dsDNA as described in Example 10, as a result of the addition of HP1 and HP2 after binding of any ssDNA sequence, it can be confirmed that a DNAzyme was effectively produced, thereby amplifying chemiluminescent and colorimetric signals about 16.5 fold or higher than the background signal of hemin and about 2.6 fold or higher than a template to which a reference DNAzyme was directly bound [FIG. 13A].

2) Confirmation of Signal Amplification

<Experimental Conditions>

DNA template (Modified OhrR_F+OhrR_R) concentration: 500 nM

H1, H2 concentration: 2 μM each

Reaction time, electrophoresis time: 30 min each

<Experimental Process>

All DNA oligomers used herein were customized by IDT. 20 μL each of 100 μM modified OhrR_F and OhrR-R were mixed, heated at 95° C. for 10 minutes, and slowly cooled at room temperature, thereby preparing an OhrR-binding double-strand sequence. Using this dsDNA as a template, 2 μL of the 5 μM template, and 2 μL each of 20 μM hairpin 1 and 20 μM hairpin 2 were used, and reacted at room temperature for 30 minutes, such that the total volume was adjusted to 20 μL. Afterward, using 8 μL of each amplified sample, agarose gel electrophoresis was performed and then a DNA band which was increased in length was visually confirmed by UV.

<Experimental Result>

As shown by the results of electrophoresis, HP1 and HP2 bands were observed at less than 50 bp in a single strand form, and a chain reaction did not occur without a template (no change in band position on DNA gel). However, in the presence of a template, H1 was first bound with the template, the hairpin structure of H1 was loosened, and the H1 sequence opened thereby was bound to the H2 sequence, resulting in obtaining an open structure of H2. As shown in the result of the reaction between the template and H1 alone, it is observed at a larger size than each of the band sizes of template DNA and H1 DNA and present in a relatively larger amount than the template DNA, and therefore, it can be observed that unbound H1 still remains below. However, when the template DNA reacted with H2 alone, they were not bound with each other, and therefore it can be confirmed that there was no change in band size of both the template and H2. In addition, it was confirmed that, in the presence of the template DNA, H1 and H2, due to a chain of template DNA binding by H1/H2, the DNA band size was effectively amplified [FIG. 13B]. Since a DNAzyme was included in the amplified DNA sequence, as shown in FIG. 13A, it was observed that DNAzyme activity was high. By using the above-described method, the DNAzyme activity was amplified through DNA-OhrR interaction by a LMW biothiol, and thus a low concentration of LMW biothiol can be effectively measured.

### Example 12: Manufacture of Biochip

A biochip method is for measuring DNA using His6-tag of OhrR, rather than by labeling with fluorescence or a DNAzyme, according to a principle that OhrR is detached from dsDNA attached to the surface of a biochip in the presence of LMW biothiols, thereby reducing a detection signal.

150 μL of 5 mg/mL NeutrAvidin® (Thermo Scientific, USA) diluted with a coating buffer (100 mM Na₂HPO₄, 50 mM citric acid) was put into each well of a transparent 96-well plate, and reacted at 37° C. for 4 to 5 hours, and then an unbound residue was washed with TBS four times. After washing, 300 μL of a Seablock blocking buffer (Thermo Scientific, USA) was put into each well and shaken at room temperature for 2 hours for blocking (process of coating NeutrAvidin-unbound surface to prevent non-specific reaction), followed by re-washing with TBS four times. 100 μL of a biotin-DNA solution diluted with a TBS buffer to 1 μM was put into each well of the blocking-completed microplate, and reacted at room temperature for 1 hour, followed by washing with TBS four times. Afterward, 100 μL of 5 μM his6-tag-conjugated OhrR was added for binding to the DNA at room temperature for 1 hour, and the resulting product was washed with TBS four times, thereby preparing a biochip for measuring LMW biothiols.

50 μL of one of CHP (final 5 μM) and a LMW biothiol (final 100 μM for each of L-cysteine, homocysteine and GSH) was simultaneously put into each well using the biochip to which biotin-dsDNA and His6-tag OhrR were fixed to the NeutrAvidin-coated plate, reacted at room temperature for 10 minutes, and washed with TBS for 4 times. Afterward, 100 μL of a HisProbe-horseradish peroxidase conjugate (HisProbe-HRP, Thermo Scientific, USA; HRP is a horseradish peroxidase) solution was added and bound at room temperature for 1 hour, washed with TBS four times, and then treated with a 3,3′, 5,5′-tetramethylbenzidine (TMB) solution. After 5 minutes, 2M sulfuric acid was added to stop the reaction, and absorbance was measured at 450 nm. Afterward, the results were compared [FIG. 14].

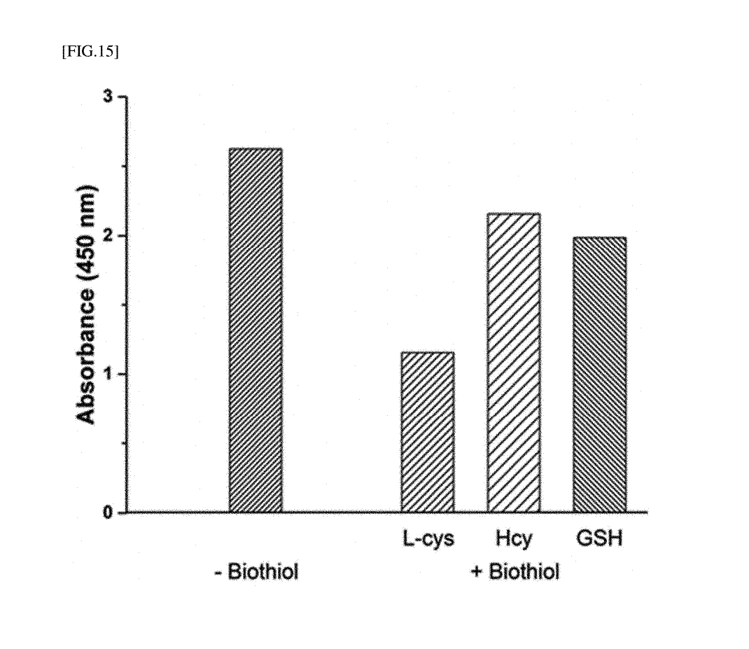

In a well in which a LMW biothiol was not present, as OhrR was bound to dsDNA fixed to the well, an absorbance signal of TMB reacting with HRP to which Hisprobe-HRP targeting His-tag of OhrR was bound was the highest. However, in the presence of a LMW biothiol and CHP, OhrR was dissociated from dsDNA fixed to the surface of a biochip, and removed by washing. Therefore, an absorbance signal of TMB reacting with HRP by binding of Hisprobe-HRP was reduced. As shown in Example 9, it was observed that, under the same concentration condition, a type of LMW biothiol such as L-cysteine exhibited a higher absorbance signal reducing effect than another type of LMW biothiol such as homocysteine or GSH [FIG. 15].

### Example 13: Manufacture of Strip-Type Biosensor

A strip sensor illustrated in FIG. 16 may consist of three major parts (sample introduction part, reaction part, and measurement part), as a membrane for manufacturing the strip sensor, cellulose, nitrocellulose or a glass-fiber membrane may be used. The structure and characteristics of each part are as follows.

Each part consists of a different membrane, and each membrane is fixed onto a universal OHP film (0.4 cm×5.5 cm) in an overlapping manner. A pad of the reaction part is placed on the lowermost region to constantly maintain the capillary phenomenon of the entire strip sensor, and fixed to be connected to the sample introduction part and the measurement part, and the sample introduction part is placed on the uppermost region to facilitate absorption.

Sample introduction part (corresponding to the left black part of the sensor in the upper panel of FIG. 16 where sample is introduced): 10 μL of a solution (TBS buffer) containing 1 μM of a FLAG tagged OhrR and dsDNA (SEQ ID NOs: 1 and 2)-bound complex and 10 μL of a LMW biothiol-containing sample solution (buffer, blood or urine) are mixed, 20 μL of the mixture is reacted with 20 μL of a buffer including a CHP solution (2 μM), and a total of 40 μL of the solution is dropped on a cellulose membrane (0.4 cm×1.5 cm) and adsorbed, such that a material dissolved in the sample migrates to the sensor reaction part according to the principle of chromatography.

Reaction part (corresponding to the central gray part, where DNA migration are drawn with arrows, of the sensor of the upper panel of FIG. 16): Consisting of a nitrocellulose membrane (about 0.4 cm×2.5 cm), about 1 μL of an anti-FLAG antibody solution (1 mg/mL, phosphate buffered solution) is dropped about 1 cm to the right from the sample introduction part, and immobilized by being left at 37° C. for 1 hour. The Flag-OhrR-dsDNA complex reacted in the sample introduction part has migrated to this part and is bound by a FLAG antibody fixed to its surface. When a LMW biothiol is present in a sample, dsDNA is dissociated from OhrR and keeps migrating on the membrane to the right, and when a LMW biothiol is not present, dsDNA is fixed like OhrR by an antibody, and migration stops.

Measurement part (corresponding to the right black part of the sensor shown in the upper panel of FIG. 16 for confirming whether LMW biothiols are detected): The measurement part is a part for measuring dsDNA dissociated from binding and separation parts and migrated, and inducing a signal of a DNAzyme attached to the end of dsDNA. In other words, a hemin solution is previously dispensed to remain on a glass-fiber membrane (0.4 cm×0.5 cm), and a final TMB solution or an ECL solution is dropped, thereby confirming a signal response. Hemin is prepared by adding a 5% casein solution to a buffer (40 mM Tris, 200 mM NaCl, 50 mM KCl and 20 mM MgCl₂) solution, and the glass-fiber membrane is sufficiently absorbed with 100 μL of hemin and dried at 55° C. for 30 minutes. After the dsDNA dissociated in the reaction part has sufficiently migrated, for final signal analysis, 10 μL of each of an ECL solution and a TMB reaction solution is added to induce a reaction. The ECL solution is imaged and analyzed using a chemiluminescence analyzer immediately after addition to the reaction solution, and the TMB reaction result can be analyzed on a mobile phone or digital camera-installed station about 30 minutes after the addition of a solution.

One sample is divided into two with the same volume and one is treated with DTT for 1 hour or longer and reacts with a DNA-OhrR complex, and the other reacts with a DNA-OhrR complex without DTT addition. In other words, the following parameters can be determined from results of two equal analyses.

Sample 1: DTT not added, measurement of free LMW biothiols

Sample 2: DTT added, measurement of total LMW biothiols

The reaction results of Samples 1 and 2 were analyzed and compared according to characteristics of each sample, disease type, disease stage, age and sex.

1. A LMW biothiol detecting composition comprising a redox-regulating protein.

2. The biothiol detecting composition according to claim 1, wherein the redox-regulating protein is one or more selected from the group consisting of an organic hydroperoxide regulator protein (OhrR), a peroxide regulator (PerR) and an oxygen regulator (OxyR).

3. The LMW biothiol detecting composition according to claim 1, wherein the redox-regulating protein includes its mutants which are capable of regulating the binding affinity between free LMW biothiol and operator dsDNA, or an affinity tagged-redox-regulating protein.

4. The LMW biothiol detecting composition according to claim 1, further comprising:
DNA binding to the redox-regulating protein.

5. The LMW biothiol detecting composition according to claim 4, wherein the DNA is bound with a fluorescent factor or a DNA-based enzyme (DNAzyme), or prepared by amplifying the DNA sequence by a signal amplification method.

6. The LMW biothiol detecting composition according to claim 5, wherein the fluorescent factor is one or more selected from the group consisting of rhodamine and a derivative thereof, fluorescein and a derivative thereof, coumarin and a derivative thereof, acridine and a derivative thereof, pyrene and a derivative thereof, erythrosine and a derivative thereof, eosin and a derivative thereof, and 4-acetamido-4′-isothiocyanatostilbene-2,2′-disulfonic acid.

7. The LMW biothiol detecting composition according to claim 5, wherein the DNA-based enzyme has one or more sequences selected from the group consisting of a peroxidase-mimicking DNAzyme and an RNA-cleaving DNAzyme.

8. The composition according to claim 1, wherein free and total LMW biothiols are individually or simultaneously detected.

9. The LMW biothiol detecting composition according to claim 8, further comprising a reducing agent when the total LMW biothiol is detected.

10. The LMW biothiol detecting composition according to claim 9, wherein the reducing agent is one or more selected from the group consisting of dithiothreitol (DTT), 2-mercaptoethanol and tris(2-carboxyethyl)phosphine (TCET).

11. The LMW biothiol detecting composition according to claim 1, wherein the biothiol has a molecular weight of 10 to 1000 Da.

12. The LMW biothiol detecting composition according to claim 1, wherein the biothiol is one or more selected from the group consisting of cysteine (Cys), homocysteine (Hcy), glutathione (GSH), N-acetylcysteine (NAC), cysteamine (CA), γ-glutamylcysteine (γ-GluCys), cysteinylglycine (CysGly), N-acetylcysteine (N-AC), coenzyme A (CoA), coenzyme B (CoB), coenzyme M (CoM), bacillithiol (BacT), mycothiol (MyT), ergothioneine (ErT), and trypanothione (TrT).

13. The LMW biothiol detecting composition according to claim 1, wherein the biothiol is related to major diseases including a cardiovascular disease, a neurodegenerative disease, cancer, kidney dysfunction, diabetes mellitus, and bacterial and viral infections.

14. A method of detecting LMW biothiols using the biothiol detecting composition of claim 1.

15. The method according to claim 14, wherein one or more selected from the group consisting of gel electrophoresis, fluorescence anisotropy, Matrix-Assisted Laser Desorption Ionization-Time-of-Flight Mass Spectrometry (MALDI-TOF MS), surface plasmon resonance (SPR), interferometry, and a bead measurement method is used.

16. A biosensor for detecting LMW biothiols, comprising the biothiol detecting composition of claim 1.

17. The biosensor according to claim 16, which is a strip type.

18. A biochip for detecting LMW biothiols, comprising the biothiol detecting composition of claim 1.

19. The biochip according to claim 18, wherein a redox-regulating protein in the LMW biothiol detecting composition has an affinity tag.

20. The biochip according to claim 18, wherein the redox-regulating protein in the LMW biothiol detecting composition forms a protein complex with DNA on a plate on which DNA that is able to be bound with the protein is immobilized.

21. A strip-type biosensor for detecting a biothiol, comprising:
a sample introduction part which includes a fixing part capable of binding to a complex of a redox-regulating protein and DNA binding to the protein, and introduces a mixture of a sample and the complex;
a reaction part linearly spaced a predetermined distance apart from the sample introduction part, and in which DNA is dissociated after oxidation of the complex; and
a measurement part formed to measure LMW biothiols by transferring the dissociated DNA.

22. The biosensor according to claim 21, wherein an antibody or receptor that is able to recognize the redox-regulating protein part binds to the fixing part.


SEQUENCE LISTING (12 sequences)

SEQ ID NO: 1 - DNA, 30 bp, Artificial Sequence
  Template DNA
tacaattaaattgtatacaattaaattgta

SEQ ID NO: 2 - DNA, 30 bp, Artificial Sequence
  Complemenatary DNA
tacaatttaattgtatacaatttaattgta

SEQ ID NO: 3 - protein, 147 aa, Bacillus subtilis
MENKFDHMKLENQLCFLLYASSREMTKQYKPLLDKLNITYPQYLALLLLWEHETLTVKKMGEQLYLDSGTLTPMLKRMEQQGLITRKRSEEDERSVLISLTEDGALLKEKAVDIPGTILGLSKQSGEDLKQLKSALYTLLETLHQKN

SEQ ID NO: 4 - DNA, 56 bp, Artificial Sequence
  Template DNA binging DNAzyme
gggttgggcgggatgggttttttttttacaattaaattgtatacaattaaattgta

SEQ ID NO: 5 - DNA, 30 bp, Artificial Sequence; identical to SEQ ID NO: 2
  Complementary DNA

SEQ ID NO: 6 - DNA, 17 bp, Artificial Sequence
  DNAzyme
gggttgggcgggatggg

SEQ ID NO: 7 - protein, 8 aa, Artificial Sequence
  Flag peptide
DYKDDDDK

SEQ ID NO: 8 - DNA, 30 bp, Artificial Sequence; identical to SEQ ID NO: 1
  OhrR_F

SEQ ID NO: 9 - DNA, 30 bp, Artificial Sequence; identical to SEQ ID NO: 2
  OhrR_R

SEQ ID NO: 10 - DNA, 42 bp, Artificial Sequence
  Modified OhrR_F
tacaattaaattgtatacaattaaattgtaaccacccaccgg

SEQ ID NO: 11 - DNA, 45 bp, Artificial Sequence
  Hairpin 1
accacccaccgggggtgggtcgtctgccggtgggtggttacaatt

SEQ ID NO: 12 - DNA, 61 bp, Artificial Sequence
  Hairpin 2
cagacgacccacccccggtgggtggtaattgtaaccacccaccgggggtgggtgggtgggt